(19) United States
(12) Patent Application Publication
(10) Pub. No.: US 2013/0253571 A1
(43) Pub. Date: Sep. 26, 2013

(54) APPARATUS AND METHODS FOR FILTERING EMBOLI DURING PERCUTANEOUS AORTIC VALVE REPLACEMENT AND REPAIR PROCEDURES WITH FILTRATION SYSTEM COUPLED IN-SITU TO DISTAL END OF SHEATH

(71) Applicant: Mark C. Bates, Encinitas, CA (US)

(72) Inventor: Mark C. Bates, Encinitas, CA (US)

(73) Assignee: NEXEON MEDSYSTEMS, INC., Charleston, WV (US)

(21) Appl. No.: 13/802,374

(22) Filed: Mar. 13, 2013

Related U.S. Application Data
(60) Provisional application No. 61/613,890, filed on Mar. 21, 2012.

(51) Int. Cl. A61F 2/01; A61F 2/24

(52) U.S. Cl. CPC A61F 2/013; A61F 2/2427
USPC 606/200

(57) ABSTRACT

Embodiments of the present invention provide apparatus and methods for embolic filtering during percutaneous valve replacement and repair procedures. Under one aspect, an apparatus comprises a sheath and a filter. The sheath has proximal and distal ends and a lumen therebetween. The distal end may be introduced into the aortic arch via the peripheral arteries and ascending aorta, while the proximal end may be disposed outside of the body. The lumen permits percutaneous aortic valve replacement or repair therethrough. The filter has a frame with an inlet and an outlet and an emboli-filtering mesh attached to the frame. The inlet is substantially spans the aortic arch in a region between the aortic valve and the great arteries. The outlet couples to the distal end of the sheath without leaving any gaps through which emboli could pass and without obstructing the lumen at the distal end of the sheath.

### CROSS-REFERENCE TO RELATED APPLICATIONS

This application claims the benefit of priority of U.S. Provisional Application Ser. No. 61/613,890, filed Mar. 21, 2012.

### FIELD OF THE INVENTION

This application generally relates to filtering emboli during interventional procedures, particularly percutaneous aortic valve replacement and repair procedures.

### BACKGROUND OF THE INVENTION

The recent development of prosthetic valves that can be placed through a catheter into the heart without thoracotomy represents a significant advance in the field of cardiovascular medicine. Early results are very promising and overall reduction in mortality has been achieved with transcatheter aortic valve implantation (TAVI) in high surgical risk patients when compared to medical therapy. One of the limitations for wide acceptance of this technology is the inherent risk of embolic complication during valve access, dilation and implantation. For example, each guidewire, introducer, balloon, cutter, or prosthetic valve that is introduced into the heart via the peripheral arteries and the ascending aorta may inadvertently dislodge one or more emboli, e.g., fragments of unstable plaque, irregular atherosclerotic calcified lesions, or mural thrombus, from the aortic arch, the area surrounding the aortic valve, or the chambers of the heart. The great vessels, which branch off the greater curve of the aortic arch, may transport such emboli to vulnerable locations like the eyes and brain causing stroke or blindness. In addition embolic material can flow past the arch and occlude vessels to the spinal cord causing paralysis, to the bowel causing life threatening mesenteric ischemia/infarction, or to the renal vessels causing kidney failure, for example.

Numerous filters have been developed with the purpose of preventing emboli from entering the great vessels, particularly the carotid artery. For example, U.S. Pat. No. 8,062,324 to Shimon et al. describes a filter that is supported by a skeleton having a horizontal plane, and that is pressed against the upper portion of the aortic arch by one or more bows so as to filter any blood passing into the great arteries. Shimon describes that the filter may be inserted using a catheter. However, Shimon does not disclose how to remove the filter in such a manner as to prevent filtered emboli from re-entering the blood stream, nor so as to prevent additional emboli from being dislodged by the edges of the skeleton or the bows during removal. Additionally, if additional devices are percutaneously introduced via the ascending aorta, such devices may scrape against the filter and thus potentially cause trauma to the aortic wall or dislodge emboli from the filter. In any such device designed to deflect particles by resting on the greater curve of the arch there is also the issue of device interaction and entanglement since the typical valve is a high profile stiff catheter that will have significant outward bias along the greater curve during advancement across the arch. This type of interaction could result in marriage of the devices together with catastrophic consequences as well as product incompetence if it folds up during catheter exchanges.

U.S. Pat. No. 8,052,713 to Khosravi et al. describes an apparatus for filtering emboli from the ascending aorta, that includes a thin, flexible, blood permeable sac having a mouth defined by a support hoop affixed to a guide wire, and a relatively short delivery sheath with a tapered proximal nose and a square distal end. Khosravi describes that the sac and support hoop may be disposed in the delivery sheath, which may be introduced to the ascending aorta via a guidewire. Khosravi describes that the sac may be deployed in the ascending aorta by retracting the support hoop proximally relative to the delivery sheath (in the direction away from the tapered nose), which draws the hoop out of the sheath and allows the sac to open across the aorta, proximal of the brachiocephalic trunk. Khosravi describes that the sac may be retrieved by advancing the support hoop back into the delivery sheath to collapse the sac, and then retracting the delivery sheath back down the ascending aorta. However, the square distal end of the delivery sheath may scrape the aortic arch as it is retrieved and thus potentially loosen additional occlusive material, such as emboli, from the aortic arch. Additionally, because the sac spans the aorta when deployed, the sac may impede the physician's ability to percutaneously introduce other devices to the aorta because such devices may become trapped in the sac, or alternatively may create a gap between the edge of the sac and the aortic wall, thus providing an avenue for occlusive material to bypass the sac.

Thus, there is a need in the art for embolic filters that may be deployed in the ascending aorta, that safely sequester any filtered occlusive material such as emboli, are shaped to avoid dislodging additional occlusive material from the vessel walls when retrieved, provide protection during all stages of the procedure and allow percutaneous valve replacement or repair procedures to be performed via the peripheral arteries and the ascending aorta without increasing the profile of the delivery sheath, which already may be at the limits of femoral vessel tolerance.

### SUMMARY OF THE INVENTION

Embodiments of the present invention provide apparatus and methods for filtering occlusive material such as emboli or thrombus during percutaneous valve replacement and repair procedures. Such apparatus and methods may safely sequester any filtered emboli, are shaped to avoid dislodging additional emboli when retrieved, are fully compatible with percutaneous valve replacement or repair procedures performed via the peripheral arteries and the ascending aorta, and do not require use of a delivery sheath larger than those already adopted for such percutaneous procedures (e.g., 18 French).

Under one aspect of the present invention, an apparatus for filtering emboli during a percutaneous aortic valve replacement or repair procedure comprises a sheath and a filter. The sheath has proximal and distal ends and a lumen therebetween. The distal end is configured for introduction into the aortic arch via the peripheral arteries and ascending aorta, while the proximal end being configured to be disposed outside of the body. The lumen is sized to permit percutaneous aortic valve replacement or repair therethrough. The filter has a frame and an emboli-filtering mesh attached to the frame. The frame has an inlet and an outlet. The inlet is configured to substantially span the aortic arch in a region between the aortic valve and the great arteries. The outlet is configured to couple to the distal end of the sheath without leaving any gaps through which emboli could pass and without obstructing the lumen at the distal end of the sheath.

In some embodiments, a plurality of tensioning lines are each coupled to the frame of the filter with the proximal portion secured to the sheath at an anchor point. In other embodiments, these tensioning lines may pass through the length of the body of the sheath and be retractable from outside of the body to draw the outlet of the filter into contact with the distal end of the sheath. A plurality of grooves may be defined in the lumen of the sheath, each groove configured to receive a corresponding tensioning line.

In some embodiments, a snare is coupled to the frame of the filter and passes out of the body through the lumen. The snare may be retractable from outside of the body to draw the filter into the lumen. A groove may be defined in the lumen of the sheath and configured to receive the snare. A leverage member may be disposed between the frame of the filter and the lumen, with the snare passing through the leverage member. The leverage member may be configured to close the filter when the snare is retracted from outside the body before the filter is drawn into the lumen.

In some embodiments, the frame comprises a distal, generally cylindrical ring defining the inlet and/or a proximal, generally cylindrical ring defining the outlet. The frame further may comprise a plurality of struts between the rings defining the inlet and the outlet.

In some embodiments, the sheath has an inner diameter of 18 French or less. The outlet of the filter may have an inner diameter that is greater than the outer diameter of the sheath. Alternatively, the outlet of the filter may have an inner diameter that is greater than an inner diameter of the sheath.

In some embodiments, the filter has a compressed state and a deployed state. The apparatus may further include a guidewire and an introducer for use in percutaneously introducing the filter and the distal end of the sheath into the aortic arch. The introducer may include a tapered distal nose, a proximal end, a guidewire lumen configured to receive the guidewire, and a recess between the distal nose and the proximal end. The recess may be configured to receive the filter in the compressed state. The introducer may be configured for insertion within the lumen at the distal end of the sheath when the filter is expanded and coupled distally. The filter then may be crimped into the recess during the manufacturing process, and as the introducer is retracted the filter then is tucked into the sheath, leaving only the distal nosecone of the introducer visible out the distal end of the sheath, while retaining the filter in the compressed state within the recess and between the introducer and the sheath. The introducer, the filter, and the distal end of the sheath may be percutaneously introducible into the aortic arch by pushing the introducer and sheath in their married position (or coupled together) over the guidewire. A control wire may be coupled to the introducer, and the control wire may be configured to keep the proximal ring of the filter coupled to the introducer while the sheath is retracted, allowing for slow deliberate expansion of the filter and avoiding traumatic sudden expansion and advancement out the end of the sheath. Alternatively, or additionally, the introducer may include a raised segment defining a secondary proximal recess of such a diameter as to secure the filter's proximal ring to the introducer by matching the thickness tolerance in between the stepped-up region to the thickness of the filter segment above the proximal ring. In such embodiments, retraction of the sheath will allow the filter to expand from the compressed state to the deployed state. The introducer may be retrievable through the outlet of the filter and the lumen of the sheath after the filter expands to the deployed state by retracting the control wire if needed. A portion of the sheath may be pre-curved to conform to the aortic arch, and the introducer may straighten the pre-curved portion of the sheath when inserted therein.

Under another aspect of the present invention, a method of filtering emboli during a percutaneous aortic valve replacement or repair procedure may include providing a sheath having proximal and distal ends and a lumen therebetween; and providing a capture mechanism on the end for coupling with a filter that is placed separately. The filter may have a compressed state and a deployed state, a frame, and an emboli-filtering mesh attached to the frame. The frame may have an inlet and an outlet, the inlet being configured to substantially span the aortic arch in a region between the aortic valve and the great arteries in the deployed state. The filter may be advanced through the previously positioned sheath via an introducer with a recess that will accommodate the filter and release wire mechanism to control expansion during exit of the filter and counterpart locking mechanism to attach to the distal sheath coupling mechanism, thus coupling the outlet of the filter to the distal end of the sheath within the aortic arch without leaving any gaps through which emboli could pass and without obstructing the lumen at the distal end of the sheath. Alternatively, the filter and introducer may be disposed at the distal end of the sheath before inserting the distal end of the sheath into the body.

### BRIEF DESCRIPTION OF DRAWINGS

FIG. 1A illustrates a perspective view of a catheter for use in percutaneous aortic valve replacement or repair including an embolic filter and sheath assembly in an expanded configuration, according to some embodiments of the present invention.

FIG. 1B illustrates a detailed perspective view of the embolic filter and sheath assembly of FIG. 1A.

FIG. 1C illustrates a cross-sectional view of the embolic filter and sheath assembly of FIG. 1A, also in an expanded configuration.

FIGS. 2A-2E illustrate various cross-sectional views of a sheath that may be used in the embolic filter and sheath assembly of FIGS. 1A-1C.

FIGS. 3A-3B illustrate perspective views of an introducer that may be used with the embolic filter and sheath assembly of FIGS. 1A-1C.

FIG. 3C illustrates a perspective view of a pre-curved sheath that may be used in the embolic filter and sheath assembly of FIGS. 1A-1C and the introducer of FIGS. 3A-3B.

FIG. 4 illustrates steps in a method of using the embolic filter and sheath assembly of FIGS. 1A-1C in an aortic arch during a percutaneous procedure.

FIGS. 5A-5C illustrate cross-sectional views of the embolic filter and sheath assembly of FIGS. 1A-1C in an aortic arch during various steps of the method of FIG. 4.

FIGS. 6A-6D illustrate perspective views of the removal of the embolic filter and sheath assembly of FIGS. 1A-1C.

FIG. 7 illustrates a perspective view of an alternative embolic filter and sheath assembly that may be used with the embolic filter and sheath assembly of FIGS. 1A-1C.

FIGS. 8A-8B illustrate cross-sectional views of another alternative embolic filter and sheath assembly that may be used with the embolic filter and sheath assembly of FIGS. 1A-1C.

### DETAILED DESCRIPTION

Embodiments of the invention provide embolic filters that readily may be used during percutaneous aortic valve replacement and repair procedures and that overcome the above-noted shortcomings of previously-known systems. The inventive filters may be compressed to a size suitable for percutaneous delivery into the aorta using a relatively small diameter sheath, e.g., an 18 F sheath, mounted on an introducer having a tapered nose, and disposed in the distal end of the sheath. The sheath containing the introducer and compressed filter then is introduced to the aortic arch via the peripheral arterial system (e.g., femoral artery) and ascending aorta. The filter then is deployed from the distal end of the sheath by retracting the sheath relative to the introducer such that the filter expands to a deployed configuration at a location upstream of the great arteries, and the introducer then removed. The filter is configured to securely dock onto the distal end of the sheath in such a manner that the full lumen of the sheath then may be used for additional percutaneous procedures, e.g., to percutaneously introduce a guidewire, introducer, balloon, cutter, and/or prosthetic valve to the heart via the sheath. Tensioning lines may be used to maintain secure coupling of the filter to the distal end of the sheath during such procedures, so as to prevent emboli from escaping through gaps between the filter and the sheath and to insure symmetrical coupling while also reducing the risk of uncoupling or separation. Then, when the percutaneous procedure is complete and any other devices have been removed from the lumen of the sheath, a snare on the filter may be used to close the filter and retract the filter and any captured emboli into the lumen of the sheath after venting the sheath. The sheath then may be removed by retracting it from the ascending aorta and peripheral arterial system. As such, the inventive filters do not interfere with other percutaneously introduced devices, are compatible with 18 French sheaths, safely sequester filtered emboli when removed, and are shaped to avoid dislodging additional emboli when removed.

First, an overview of a catheter system including the inventive embolic filter and sheath assembly will be described. Then, further details will be provided on the construction of the sheath and embolic filter, respectively. Lastly, some alternative embodiments will be described.

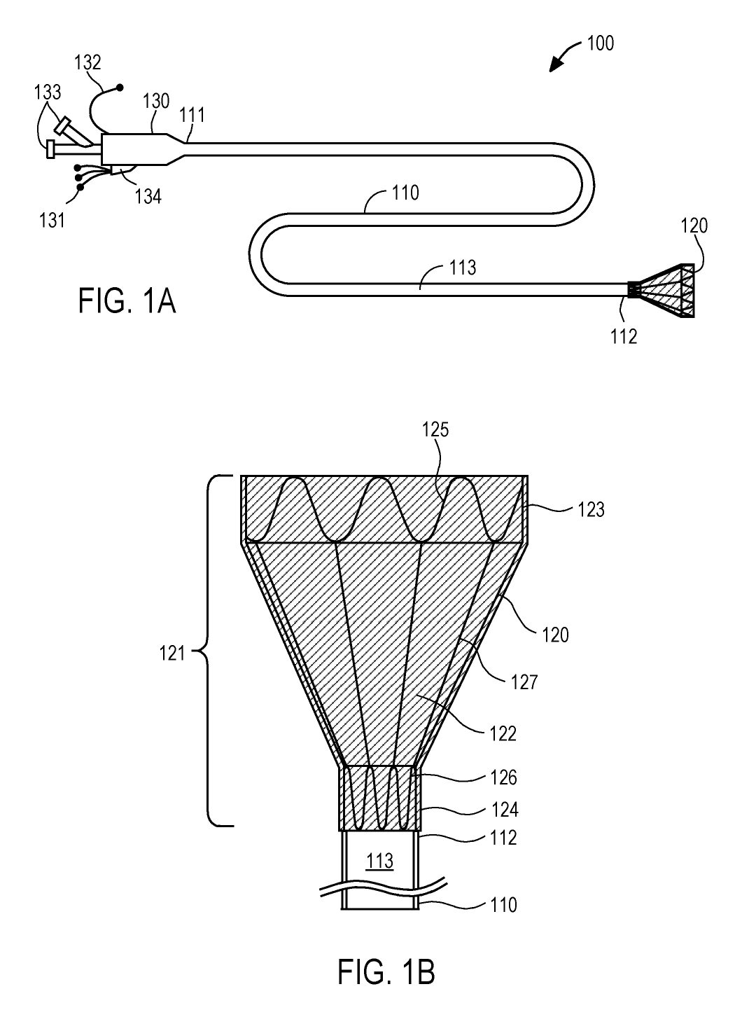

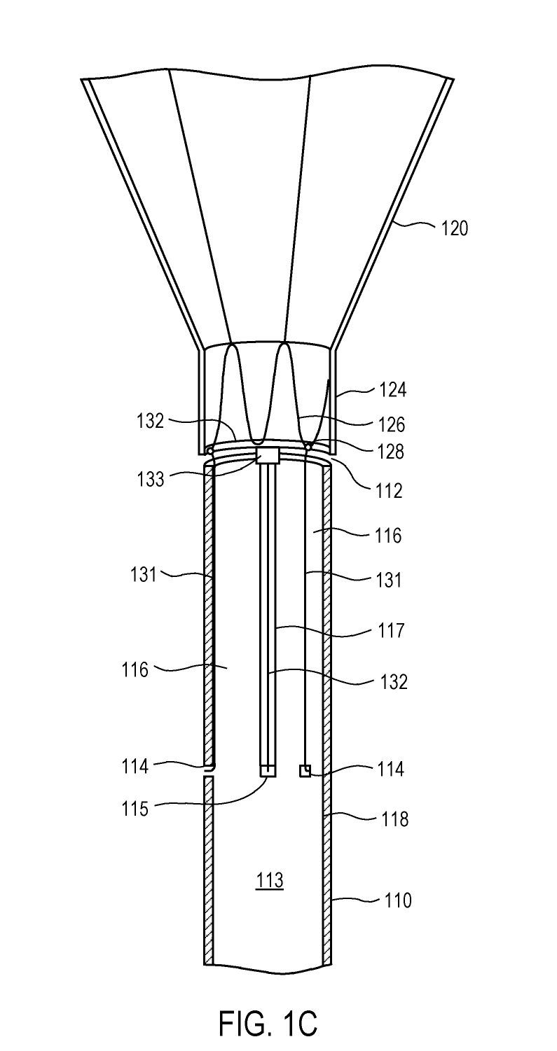

FIG. 1A illustrates percutaneous catheter 100 including sheath 110, filter 120, and handle 130. Sheath 110 generally is in the form of an elongated tube having proximal end 111 and distal end 112, with lumen 113 therebetween. Preferably, sheath 110 has an outer diameter suitable for percutaneous use, e.g., is 18 French or smaller. In some embodiments, sheath 110 includes reinforcing rings of metal or polymer to inhibit collapse of the sheath when curved around the aortic arch.

Filter 120 includes a frame and an emboli-filtering mesh attached to the frame. The frame defines an inlet and an outlet of filter 120. Preferably, the inlet has lateral dimensions approximately equal to those of the aortic arch between the aortic valve and the great arteries, where the filter will be deployed, so that the emboli-filtering mesh will filter substantially all of the blood passing through the aorta and remove emboli therefrom. The outlet of filter 120 is detachably coupled to distal end 112 of sheath 110, preferably without any gaps therebetween that would allow emboli to pass. The outlet of filter 120 also preferably has an inner lumen with a diameter that is at least as large as the inner diameter of sheath 110, so that filter 120 does not obstruct the lumen at the distal end of the sheath, thus allowing a physician to perform percutaneous procedures via the sheath without interference from filter 110.

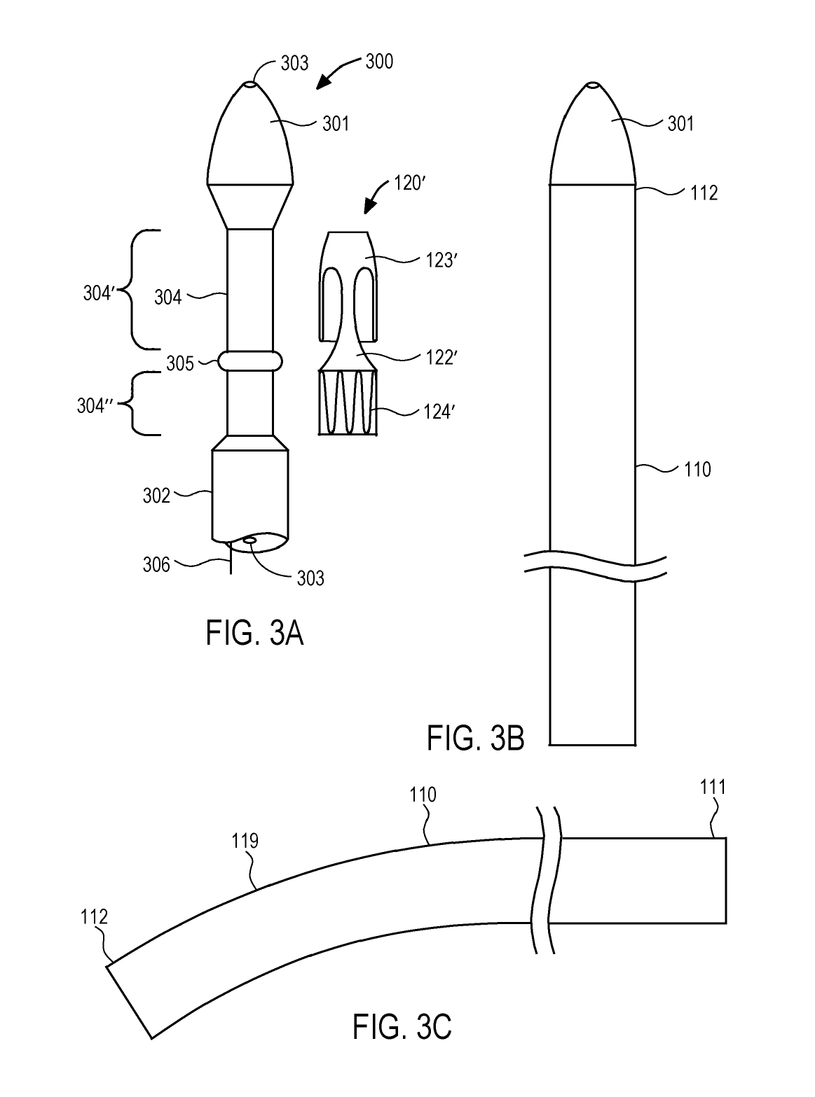

Handle 130 is coupled to proximal end 111 of sheath 110, and includes tensioning lines 131 via which filter 120 may be retracted into secure engagement with distal end 112 of sheath 110 while deployed, ratchet 134 which may be used to secure tensioning lines 131 in a retracted position, snare control 132 via which filter 120 may be retrieved by retracting the filter into the lumen at the distal end 112 of sheath 110, and various additional ports and passages, generally designated 133, via which a physician may introduce additional percutaneous devices. Handle 130 also may include a controller line (not shown) for controlling an introducer that may be used to deploy the filter, such as described below with reference to FIGS. 3A-3C.

Note that as used herein with reference to elements for insertion into the body, the term “distal” refers to the end that is inserted into the body first, e.g., the leading end of sheath 110 or filter 120 during advancement into the body, whereas the term “proximal” refers to the opposite end.

FIG. 1B illustrates a perspective view of an assembly including filter 120 and distal end 112 of sheath 110. Filter 120 includes frame 121 and mesh 122 attached thereto, e.g., by sutures, adhesives, dip molding, laser bonding, sandwich layers on each side of the struts melted or glued together, heat setting or the like. In the illustrated embodiment, frame 121 includes first and second generally cylindrical rings 123, 124. First ring 123 defines the inlet of filter 120, which as noted above preferably is of similar dimension to the ascending aorta in the region where the filter is to be deployed, e.g., between the aortic valve and the great arteries, so as to securely seat against the aortic wall and prevent emboli from slipping past the filter. Second ring 124 defines the outlet of filter 120, which is of similar dimension to distal end 112 of sheath 110. Specifically, second ring 124 is sized such that it does not obstruct lumen 113 of sheath 110 at distal end 112, so that the physician may conduct percutaneous procedures through lumen 113 without interference from filter 120. For example, second ring 124 may have an inner diameter that is equal to, or larger than, the inner diameter of lumen 113. Or, for example, second ring 124 may have an inner diameter that is larger than the outer diameter of sheath 110, so that second ring 124 seats over the outer surface of sheath 110 when retracted by tensioning lines 131 described further below with respect to FIG. 1C.

First and second rings 123, 124 preferably are formed of a shape memory material, e.g., a metallic alloy such as Nitinol, stainless steel, MP35N, elgiloy or a shape memory polymer such as polyurethane or a block copolymer thereof, polyethylene terephthalate or a block copolymer thereof, polyethylene oxide or a block copolymer thereof, and the like. First and second rings 123, 124 respectively include struts 125, 126, which may be sinusoids, zigzags, or other suitable shape that permits rings 123, 124 to be radially compressed into a compressed state for delivery and to return to a deployed state when expanded in the aortic arch. Optionally, frame 121 includes struts 127 that extend between first and second rings 123, 124. Struts 127 may have any suitable shape, including linear, sinusoids, or curves, and may extend within the interior surface of mesh 122 and/or may extend outside of the exterior surface of mesh 122. In other embodiments, only mesh 122 extends between first and second rings 123, 124, allowing the rings to freely move relative to one another so as to lessen the effect of blood-flow-induced torque that otherwise may cause filter 120 to tilt relative to sheath 110 and thus form a gap through which emboli may pass.

Mesh 122 preferably covers the entire outer surface of filter 120, including first and second rings 123, 124, such that substantially all of the blood in the aorta flows through filter 120 with no gaps. Mesh 122 has a surface area and pore size suitable to allow a sufficient volume of blood to pass therethrough to maintain the patient's blood pressure in a normal range, and also to avoid pressure buildup that otherwise may rupture mesh 122. Mesh 122 may include any suitable material known in the art, including a fabric, polymer, or flexible metal having pores of appropriate size to filter emboli having diameters of, e.g., 20 μm or greater, or 50 μm or greater, or 100 μm or greater, or 150 μm or greater, or 200 μm or greater. In one illustrative embodiment, mesh 122 is a polyurethane film of thickness 0.0003 inches to about 0.0030 inches and having holes defined therethrough, e.g., circular, square, or triangular holes in a suitable size and density to permit substantially the entire aortic blood flow to pass therethrough without a detrimental amount of resistance.

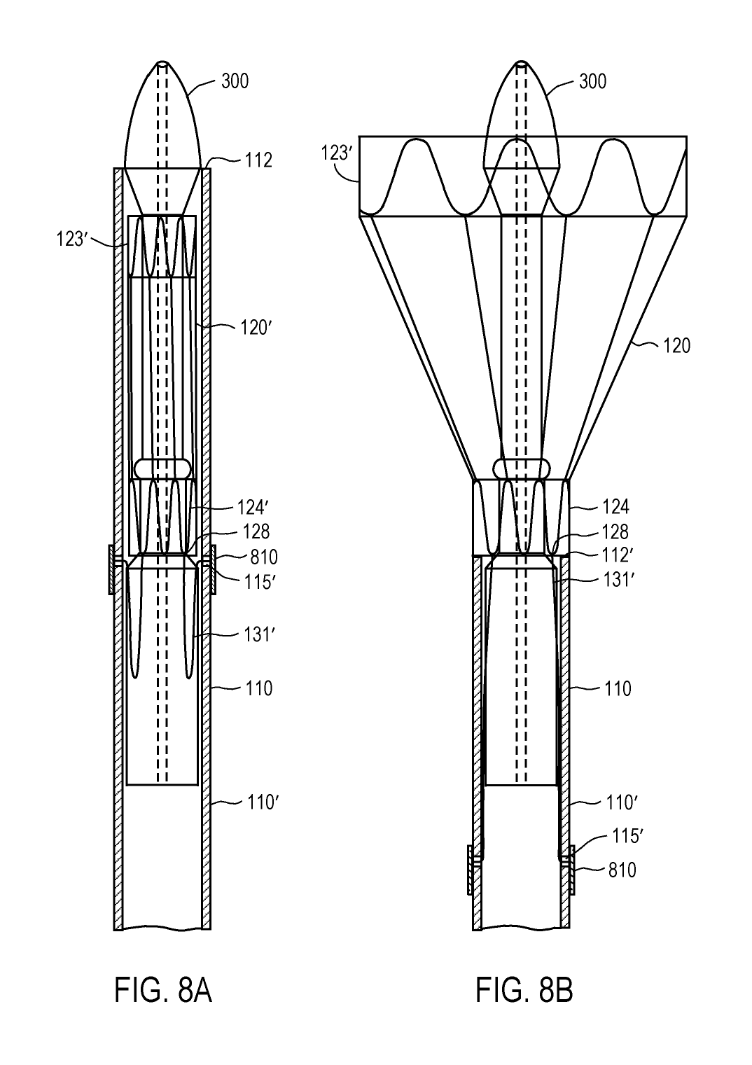

FIG. 1C illustrates additional structural detail of the coupling between distal end 112 of sheath 110 and second ring 124 of filter 120. Second ring 124 is coupled to a plurality of tensioning lines 131 that pass out of the patient's body through sheath 110 via ports 114 and into handle 130 as illustrated in FIG. 1A. For example, in the illustrated embodiment, each tensioning line 131 is respectively coupled to a minimum 128 of sinusoid 126 of second ring 124, for example via a knot or hinge. Tensioning lines 131 may be retracted to cause second ring 124 to securely seat against distal end 112. For example, each tensioning line 131 may be individually retractable via port 114 and handle 130 so as to enhance control over the seating of second ring 124 and compensate for any torque that may be placed on filter 120 due to blood flow; that is, each tensioning line 131 may be individually refracted via handle 130 to seat the portion of second ring 124 to which it is coupled against a respective portion of distal end 112 of sheath 110. Alternatively, the proximal ends of tensioning lines 131 all may be coupled to a single control line in handle 130 that may be used to uniformly retract tensioning lines 131. Preferably, tensioning lines 131 may be releasably secured once retracted so as to maintain contact between ring 124 and distal end 112, e.g., using a ratchet 134 on handle 130 illustrated in FIG. 1A or other suitable mechanism. In still another alternative embodiment described further below with reference to FIGS. 8A-8B, tensioning lines 131 may be anchored to sheath 110 at a position proximal of distal end 112.

In some embodiments, tensioning lines 131 are formed of a relatively stiff material such as stainless steel, such that tensioning lines 131 may be pushed to move filter 120 distally relative to sheath 110, as well as pulled to move filter 120 proximally relative to sheath 110. Such material is particularly useful in embodiments where the inner diameter of second ring 124 is greater than the outer diameter of sheath 110, because tensioning lines 131 may be pulled to seat second ring 124 over the outer surface of sheath 110 and later pushed to move second ring 124 off of the outer surface of sheath 110 so that filter 120 may be retracted into the lumen of sheath 110, e.g., using snare 132 described further below. In other embodiments, tensioning lines 131 may be formed of a relatively flexible material such as fiber or polymer, so that tensioning lines 131 may be pulled to seat second ring 124 against distal end 112 but pushing tensioning lines 131 has no material effect on the relative position of second ring 124 and distal end 112. In still other embodiments, tensioning lines 131 may be formed of an elastic material, e.g., an elastic polyurethane, silicon copolymer, latex, or polysiloxane modified ethylene/butylene/styrene (SEB) block copolymer or the like. Examples of materials and configurations suitable for such elastic tensioning lines may be found in U.S. Pat. No. 5,728,131, the entire contents of which are incorporated herein by reference.

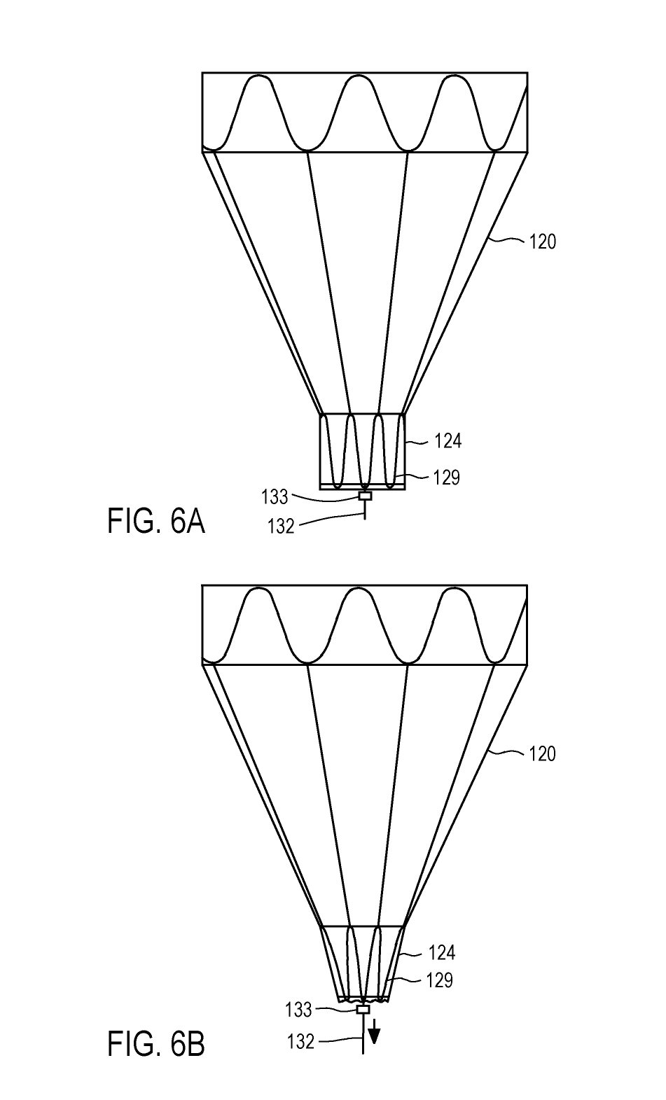

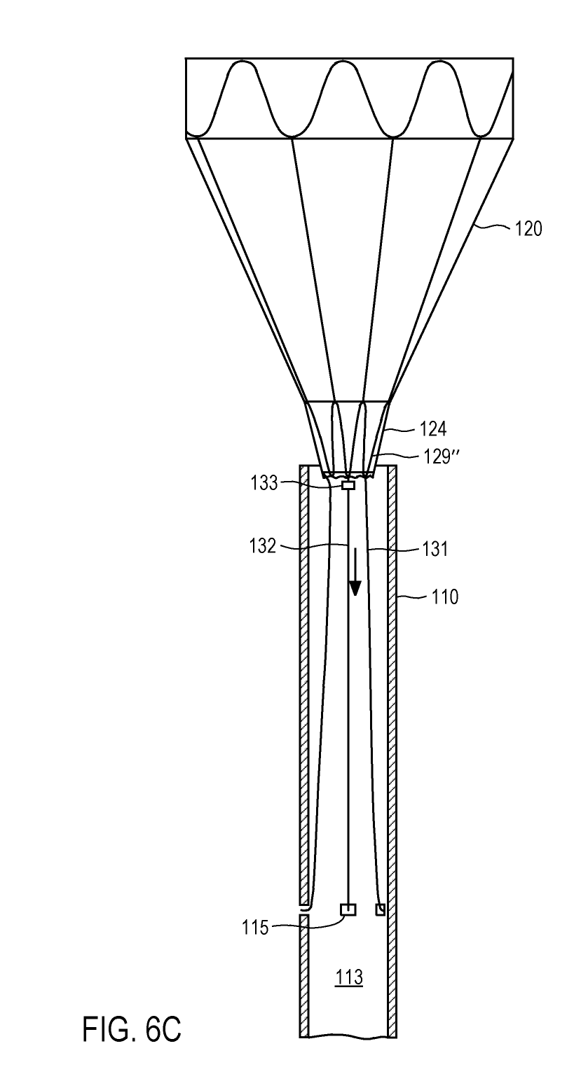

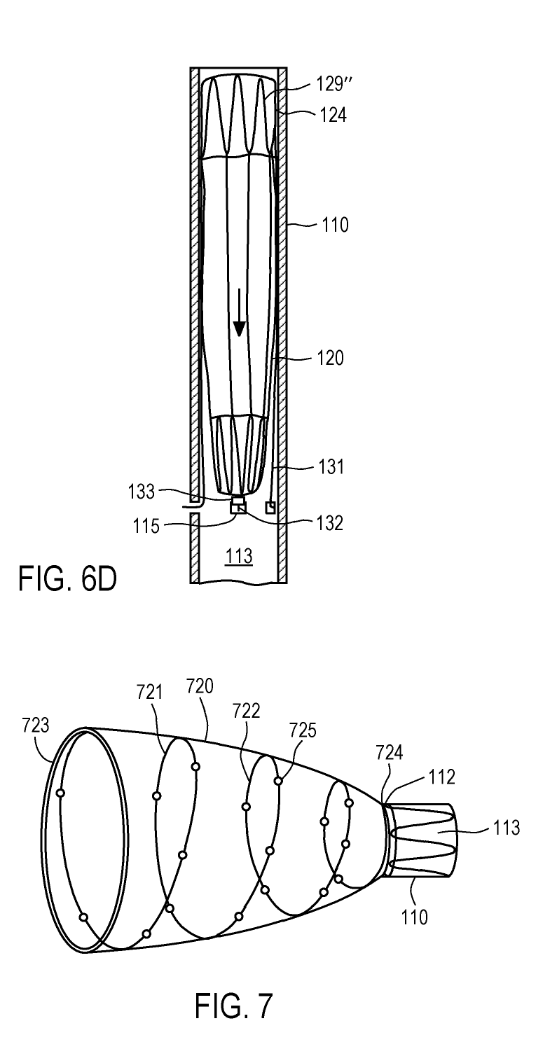

As illustrated in FIG. 1C, second ring 124 also is coupled to snare 132 that passes through leverage member 133, which is positioned between second ring 124 and distal end 112 of sheath 110. Snare 132 loops about second ring 124 and passes out of the patient's body through lumen 113 and port 114 and into handle 130 as illustrated in FIG. 1A. Retracting snare 132 via handle 130 first causes second ring 124 to radially contract as a result of leverage applied by leverage member 133, and then pulls filter 120 into lumen 113 sheath 110 for removal from the body. Such a process is described further below with reference to FIGS. 6A-6D. Alternatively, instead of providing leverage member 133 and disposing snare 132 in groove 117, leverage member 133 may be omitted and snare 132 instead disposed in a separate hypotube (not illustrated) that extends within lumen 113 of sheath 110. The distal end of the hypotube is adjacent second ring 124 and performs a similar function to that of leverage member 133. Other suitable configurations for snare 132 and leverage member 133 may be found in U.S. Pat. No. 5,713,948, the entire contents of which are incorporated herein by reference.

Tensioning lines 131 may be disposed within grooves 116 and snare 132 may be disposed within groove 117 defined in the inner surface 118 of sheath 110. Such an arrangement may inhibit interference between lines 131 or snare 132 and any devices that may be percutaneously introduced to the patient via sheath 110. In particular, the grooves 116, 117 may be of such a depth that lines 131 and snare 132 do not reduce the effective inner diameter of lumen 113, thus allowing the physician to make full use of lumen 113 without obstruction during a percutaneous procedure.

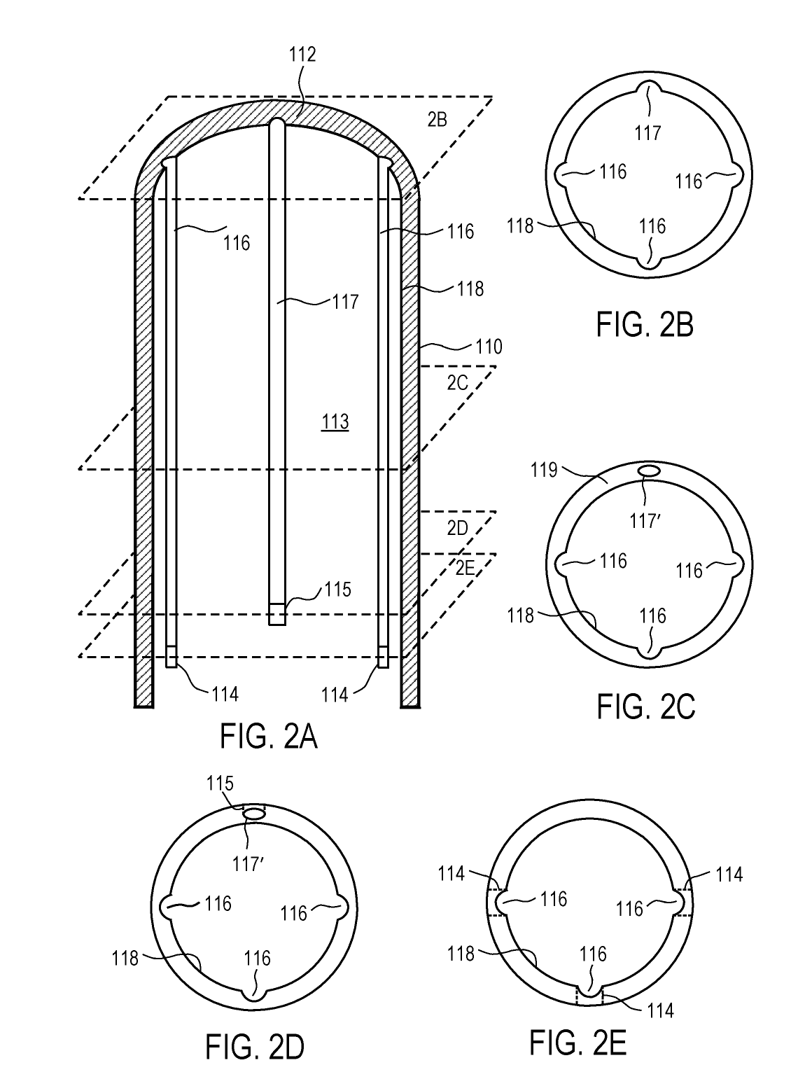

FIGS. 2A-2E illustrate an exemplary arrangement of grooves 116, 117 on inner surface 118 of sheath 110. FIG. 2A illustrates a cut-away view of the inner surface 118 of sheath 110, in which two tensioning line grooves 116 and the snare groove 117 may be seen to run substantially parallel to lumen 113 of sheath 110. FIG. 2B illustrates a cross-section of sheath 110 through plane 2B, at distal end 112 of the sheath. Grooves 116, 117 may be seen to be defined on inner surface 118 of sheath 110, with groove 117 being somewhat larger than grooves 116 because snare 132 may have a larger diameter than do tensioning lines 131. FIG. 2C illustrates a cross-section of sheath 110 through plane 2C, at a location proximal to plane 2B. Here, it may be seen that groove 117 has transitioned to lumen 117′ defined within the wall 119 of sheath 110, while grooves 116 continue along the inner surface 118 of sheath 110. FIG. 2D illustrates a cross-section of sheath 110 through plane 2D, at a location proximal to plane 2C, Here, it may be seen that port 115 is connected to lumen 117′ so as to allow passage of snare 132 out of sheath 110 and into handle 130 such as illustrated in FIG. 1A. FIG. 2E illustrates a cross-section of sheath 110 through plane 2E, at a location proximal to plane 2D. Here, it may be seen that ports 114 are connected to respective grooves 116 so as to allow passage of tensioning lines 131 out of sheath 110 and into handle 130 such as illustrated in FIG. 1A. Note that tensioning line ports 114 and snare port 115 alternatively may be in the same plane as one another, or tensioning line ports 114 may be distal relative to snare port 115. Other configurations for passing tensioning lines 131 and snare 132 along the length of, and out of sheath 110 may alternatively be used. Alternatively, as described below with reference to FIGS. 8A-8B, tensioning lines 131 may be anchored to sheath 110 at points proximal of distal end 112 instead of passing along the length of sheath 110 and into handle 130.

FIG. 3A illustrates an introducer 300 that may be used to introduce and deploy filter 120 into a patient's aortic arch. Introducer 300 includes tapered distal nose 301, proximal end 302, a guidewire lumen 303 configured to receive a guidewire (not illustrated in FIG. 3A), and a recess 304 between the distal nose and proximal end. Recess 304 is configured to receive filter 120 in a compressed state. For example, as illustrated in FIG. 3A, compressed state filter 120′ may include radially compressed first ring 123′, radially compressed second ring 124′, and folded mesh 122′, a portion of which is tucked underneath the compressed first ring so as to reduce the compressed length and diameter of compressed state filter 120′. Recess 304 of introducer 300 may be configured to have a length approximately equal to the length of compressed state filter 120′. Optionally, recess 304 also may include a raised segment 305 configured to engage a gap in filter 120′ between first and second rings 123′, 124′ so as to inhibit sliding of filter 120′ in the proximal or lateral directions. Such raised segment 305 may define a first proximal recess 304′ of such a diameter as to secure radially compressed first ring 123′, and a second proximal recess 304″ of such a diameter as to secure radially compressed second ring 124′. Preferably, recess 304 has a depth sufficient to accommodate the diameter of compressed state filter 120′ such that introducer 300 may be inserted within lumen 113 at the distal end 112 of sheath 110, where recess 304 retains compressed state filter 120′ within the recess and between introducer 300 and sheath 110. Radiopaque markers may be provided on introducer 300 and/or on filter 120/120′ so as to assist the physician in properly positioning introducer 300 and filter 120/120′ in the aortic arch.

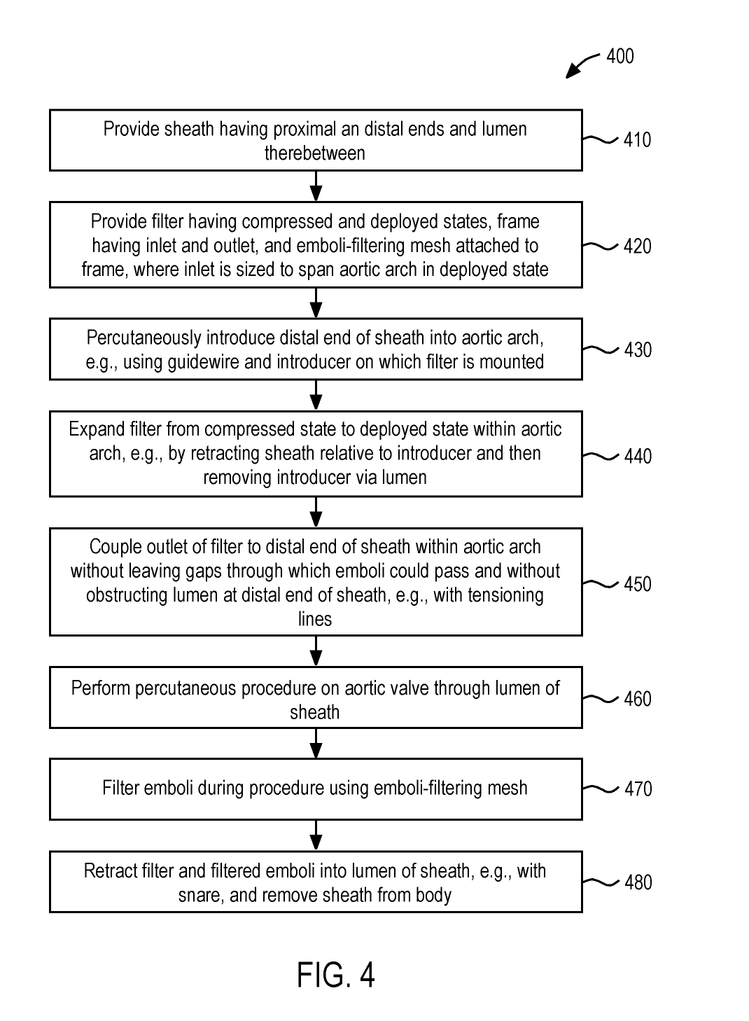

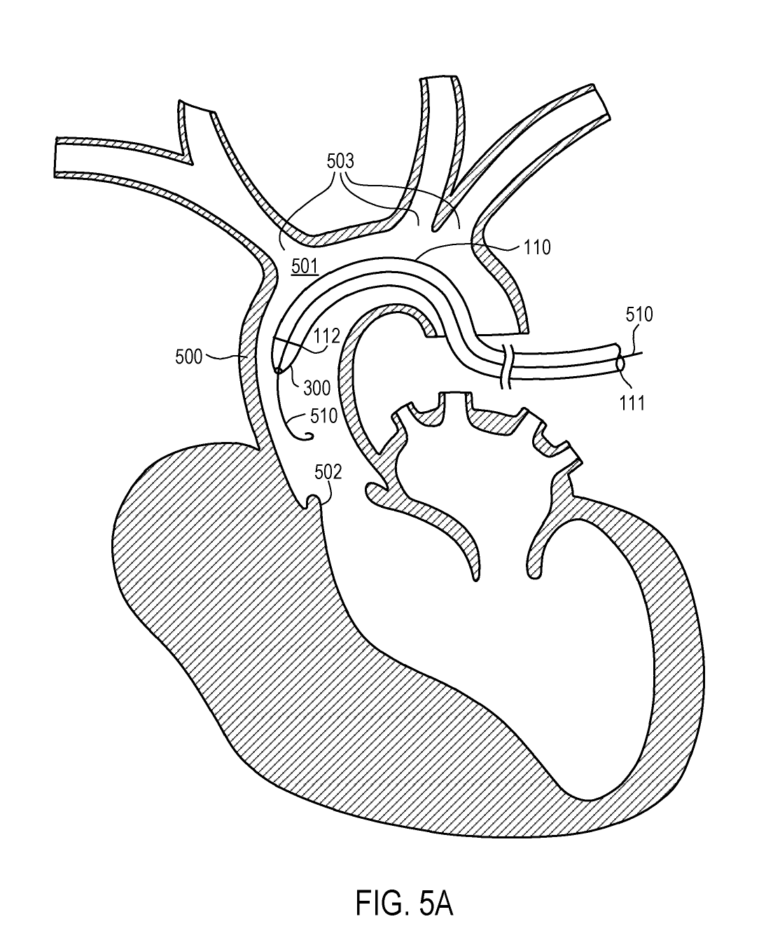

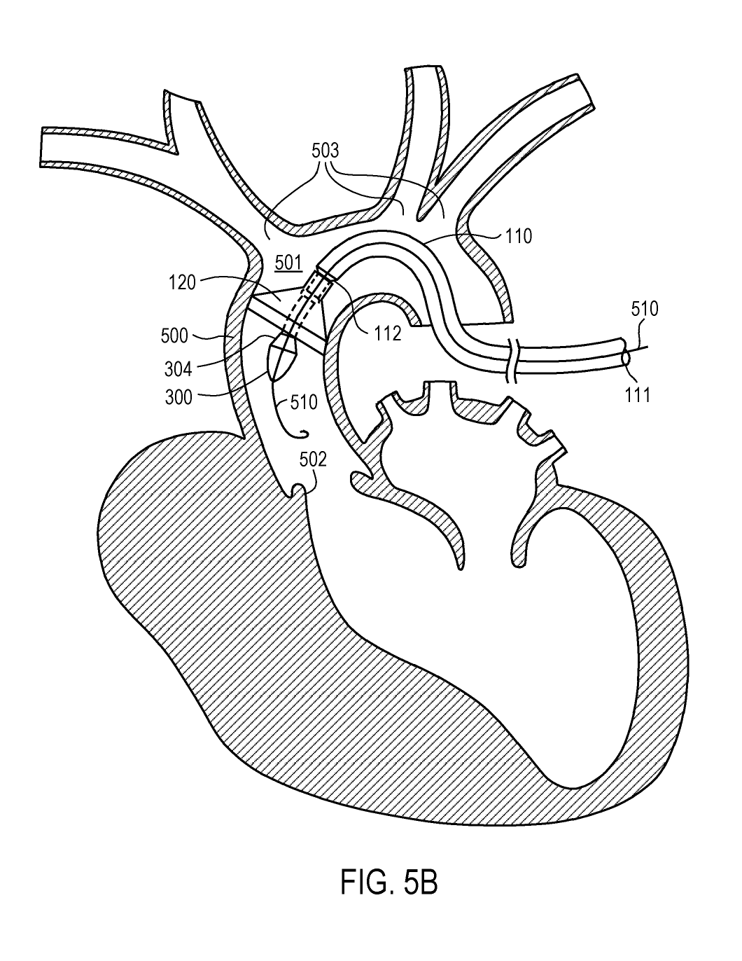

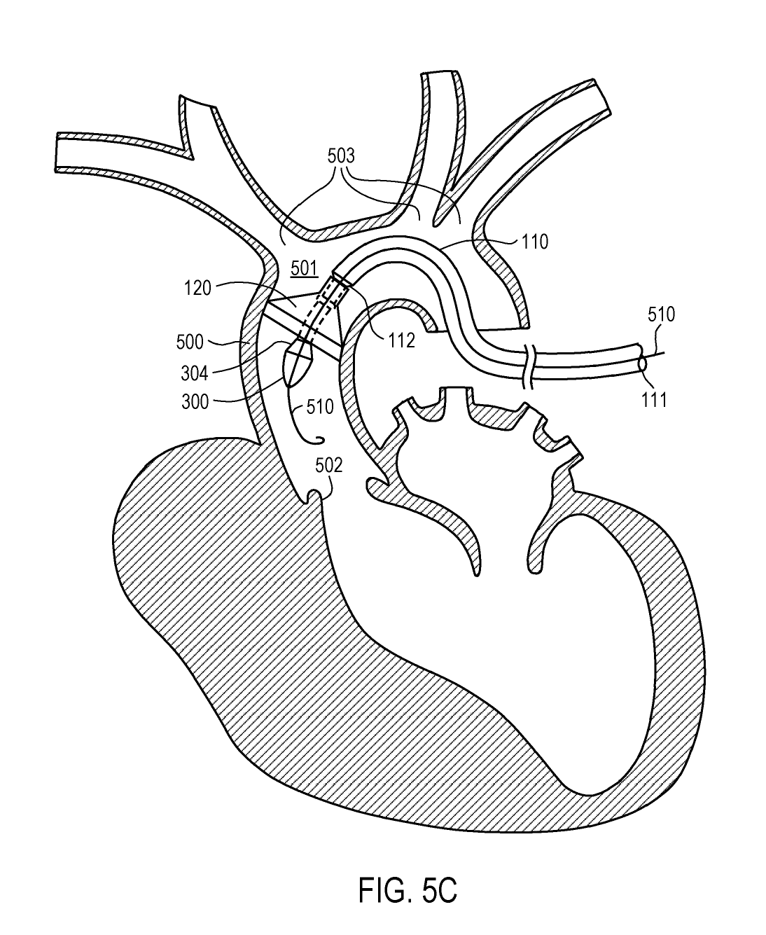

As illustrated in FIG. 3B, when introducer 300 is so inserted into sheath 110, tapered nose 301 extends past distal end 112 of sheath 110 so as to provide a smooth surface when introducer 300, compressed state filter 120′, and distal end 112 of sheath 110 are percutaneously advanced into the aortic arch by pushing introducer 300 over the guidewire (not illustrated) via sheath 110. Preferably, introducer 300 also includes control line 306 which is coupled to proximal end 302 and which passes out of the body via lumen 113 of sheath 110 and an appropriate port (not illustrated). Control line 306 may be used to retain introducer 300 in place while sheath 110 is retracted so as to allow compressed state filter 120′ to expand into the deployed state, e.g., filter 120 illustrated in FIGS. 1A-1B. Further details on the use of introducer 300 to deploy filter 120 are described in greater detail below with reference to FIGS. 4 and 5A-5C.

Optionally, sheath 110 is pre-curved to follow the curve of the patient's aortic arch, such as illustrated in FIG. 3C where bend 119 is disposed proximal of distal end 112. Pre-curving sheath 110 as such may help to reduce tension the aortic arch may otherwise place on sheath 110 and/or filter 120 when deployed therein. Preferably, inserting introducer 300 into sheath 110 temporarily straightens bend 119; later, when introducer 300 is removed from sheath 110, bend 119 returns and generally follows the curve of the patient's aortic arch.

A method of percutaneously deploying filter 120 and distal end of sheath 112 in the aortic arch for filtering emboli during a percutaneous procedure will now be described with reference to FIG. 4, which illustrates steps in method 400, and FIGS. 5A-5C, which illustrate the relative positions of components of apparatus 100 and a patient's heart.

Method 400 includes providing a sheath having proximal and distal ends and a lumen therebetween (step 410), for example sheath 110 illustrated in FIGS. 1A-2E.

A filter is also provided having a compressed state and a deployed state, a frame having an inlet sized to span the aortic arch in the deployed state and an outlet, and an emboli-filtering mesh attached to the frame (step 420), for example filter 120/120′ illustrated in FIGS. 1A-1C and 3A.

The distal end of the sheath then may be introduced into the aortic arch (step 430). For example, compressed state filter 120′ first may be crimped into recess 304 of introducer 300 illustrated in FIG. 3A, and introducer 300 inserted into lumen 113 at distal end 112 of sheath 110. Then, as illustrated in FIG. 5A, assembly 300, 120′, 112 may be percutaneously advanced into aortic arch 510 of a patient's heart 500 over guidewire 510 through guidewire lumen 303 of introducer 300, by pushing on the proximal end 111 of sheath 110 from outside the patient's body. Assembly 300, 120′, 112 (filter 120′ not shown in FIG. 5A) is preferably pushed to a location in aortic arch 501 that is between aortic valve 502 and great arteries 503.

Referring again to FIG. 4, the filter then may be expanded from the compressed state to the deployed state within the aortic arch (step 440). For example, as illustrated in FIG. 5B, distal end 112 of sheath 110 may be refracted from outside the patient's body while the position of introducer 300 is maintained, e.g., using control line 306 described above with reference to FIG. 3A. Such refraction of distal end 112 of sheath 110 exposes recess 304 of introducer 300 in which compressed state filter 120′ is disposed, allowing the filter to expand to deployed state filter 120 which, as illustrated in FIG. 5B, substantially spans the aortic arch between aortic valve 502 and the great arteries 503. In particular, control line 306 maintains the relative positioning of compressed second (proximal) ring 124′ of compressed filter 120′ and introducer 300, thus facilitating slow, deliberate expansion of filter 120 as sheath 110 is retracted and avoiding traumatic sudden expansion of filter 120 out of sheath 110. Introducer 300 then may be removed via lumen 113, e.g., by retracting control line 306 from outside the body.

Referring again to FIG. 4, the outlet of the deployed filter then is coupled to the distal end of the sheath within the aortic arch, without leaving gaps through which emboli could pass and without obstructing the lumen at the distal end of the sheath (step 450). For example, retraction of tensioning lines 131 from outside the body such as described above with reference to FIGS. 1C and 2A-2E may securely seat second ring 124 of deployed filter 120 against distal end 112 of sheath 110, as illustrated in FIG. 5C.

Referring again to FIG. 4, a percutaneous procedure may be performed on the aortic valve through the lumen of the sheath (460). Such percutaneous procedures may involve, for example, percutaneously introducing a guidewire, introducer, balloon, cutter, and/or prosthetic valve to the heart through sheath 110 and filter 120 as illustrated in FIG. 5C. For example, the physician may implant a prosthetic aortic valve that is specifically configured for percutaneous delivery via an 18 French sheath, such as the COREVALVE™ device manufactured by Medtronic, or the SAPIEN™ device manufactured by Edwards Lifesciences. Advantageously, filter 120, tensioning lines 131, and snare 132 do not obstruct lumen 113 of sheath 110, so that the physician may perform any desired percutaneous procedure using the full diameter of sheath 110. Filter 120 captures any emboli that may be freed from the region surrounding the aortic valve during such procedure, thus reducing the patient's risk of stroke from embolization.

Note that introducer 300 and filter 120 alternatively may be introduced into the aortic arch via the proximal end 111 of sheath 110 during the percutaneous procedure, rather than via the distal end 112 before the percutaneous procedure as described above. For example, distal end 112 of sheath 110 may be introduced to aortic arch 510 over a guidewire. Filter 120 may be crimped onto recess 304 of introducer 300, and the compressed filter/introducer assembly 120′/300 may be introduced into lumen 113 of sheath 110 via proximal end 111, and then advanced to distal end 112 by pushing control line 306. Filter 120 then may be deployed in the aortic arch and introducer 300 may be removed as described above and a percutaneous procedure performed via lumen 113.

An illustrative method of removing filter 120 and any filtered emboli from the body will now be described with reference to FIGS. 6A-6D. As illustrated in FIG. 6A, snare 132 may be looped around second ring 124 defining the outlet of filter 120, e.g., adjacent to proximal end 129 of second ring 124. Snare 132 then may pass through leverage member 133 and out of the body via groove 117, lumen 117′, and port 115 defined in sheath 110 as described further above with reference to FIGS. 1C and 2A-2E. Retraction of snare 132 in the proximal direction radially compresses proximal end 129 of second ring 124, resulting in partially compressed second ring 124″ having a generally conical shape as illustrated in FIG. 6B. Preferably, compressed proximal end 129″ of partially compressed second ring 124″ has an outer diameter that is less than the inner diameter of lumen 113 of sheath 110, so that further retraction of snare 132 in the proximal direction pulls proximal end 129″ of second ring 124″ into lumen 113 of sheath 113 as illustrated in FIG. 6C. Leverage member 133, if present, also may be pulled into lumen 113. As illustrated in FIG. 6D, further refraction of snare 132 in the proximal direction further compresses second ring 124′″ into a fully compressed removal state and pulls the ring deeper within lumen 113; pulls mesh 122′″ and any emboli therein into a compressed removal state within lumen 113; and pulls first ring 123′″ into a fully compressed removal state and pulls the ring into lumen 113. Sheath 110 then may be withdrawn from the body by pulling the sheath in the proximal direction. Advantageously, sheath 110 does not have any sharp corners that would potentially loosen emboli during such removal. Additionally, any emboli within mesh 122′″ advantageously remain within lumen 113 during the removal process, so as to further reduce the patient's chance of stroke due to embolization.

FIG. 7 illustrates an alternative filter 720 that may be used in place of filter 120 in the above-described embodiments. Filter 720 includes frame 721 which, like frame 121 of filter 120, may be formed of a shape memory alloy. Filter 720 further includes mesh 722 disposed over frame 721, and attached to frame 721 with sutures 725 or other suitable attachment mechanism. Frame 721 defines an inlet 723 and an outlet 724 of filter 720. Like ring 123 of filter 120, inlet 723 of filter 720 preferably is configured to span the aortic arch in a region between the aortic valve and the great arteries, so that mesh 722 may filter emboli from substantially all of the blood passing through the aortic arch. Like ring 124 of filter 120, outlet 724 of filter 710 preferably is configured to couple securely to distal end 112 of sheath 110 without leaving any gaps through which emboli could pass. Additionally outlet 724 of filter 710 preferably has an inner diameter that is equal to or greater than the inner diameter of lumen 113 of sheath 110, or that is equal to or greater than the outer diameter of sheath 110, so that percutaneous procedures may be performed via lumen 113 without interference from filter 720.

FIGS. 8A-8B illustrate an alternative filter/sheath/introducer configuration in which modified tensioning lines 131′ do not pass into handle 130 via lumen 113 and ports 115, as described above with reference to FIGS. 1A-2E, but instead each are anchored to sheath 110 at a point proximal of distal end 112. Specifically, FIG. 8A illustrates compressed filter 120′ crimped onto introducer 300 and disposed within modified sheath 110′ having modified ports 115′ defined therethrough at a point proximal of distal end 112, but at a location relatively close to distal end 112. Modified tensioning lines 131′ are coupled to minima 128 of compressed second (proximal) ring 124′, and pass through modified ports 115′ where anchors 810 anchor the ends of lines 131′ to modified sheath 110′. Modified lines 131′ are slack when filter 120′ is in the compressed configuration. As illustrated in FIG. 8B, when filter 120 is expanded by retracting modified sheath 110′ relative to introducer 300, modified tensioning lines 131′ are drawn taut by proximal movement of ports 115′ and anchors 810, thus securing filter 120 against distal end 112′ of modified sheath 110′. Modified tensioning lines 131′ have a length approximately equal to the distance between distal end 128′ and anchor 810, so that there is substantially no slack in lines 131′ when filter 120 is in the deployed configuration. In this embodiment, modified sheath 110′ may lack grooves 116 described above with references to FIGS. 1C-2E.

While various illustrative embodiments of the invention are described above, it will be apparent to one skilled in the art that various changes and modifications may be made therein without departing from the invention. For example, although the embodiments above have been described primarily with respect to configurations suitable for use in the aortic arch, it should be appreciated that the apparatus and methods suitably may be modified for percutaneous use in other blood vessels and for other applications including but not limited to: treatment of atherosclerotic arterial disease, aneurysmal disease and venous thrombosis. The appended claims are intended to cover all such changes and modifications that fall within the true spirit and scope of the invention.

What is claimed:

1. Apparatus for filtering emboli during a percutaneous aortic valve replacement or repair procedure, the apparatus comprising:
a sheath having proximal and distal ends and a lumen therebetween, the distal end being configured for introduction into the aortic arch via the peripheral arteries and ascending aorta, the proximal end being configured to be disposed outside of the body, the lumen being sized to permit percutaneous aortic valve replacement or repair therethrough; and
a filter having a frame and an emboli-filtering mesh attached to the frame, the frame having an inlet and an outlet, the inlet being configured to substantially span the aortic arch in a region between the aortic valve and the great arteries, the outlet being configured to couple to the distal end of the sheath without leaving any gaps through which emboli could pass and without obstructing the lumen at the distal end of the sheath.

2. The apparatus of claim 1, further comprising a plurality of tensioning lines coupled to the frame of the filter and passing out of the body through the lumen, the tensioning lines being retractable from outside of the body to draw the outlet of the filter into contact with the distal end of the sheath.

3. The apparatus of claim 2, wherein a plurality of grooves are defined in the lumen of the sheath, each groove configured to receive a corresponding tensioning line.

4. The apparatus of claim 1, further comprising a snare coupled to the frame of the filter and passing out of the body through the lumen, the snare being retractable from outside of the body to draw the filter into the lumen.

5. The apparatus of claim 4, wherein a groove is defined in the lumen of the sheath and configured to receive the snare.

6. The apparatus of claim 5, further comprising a leverage member between the frame of the filter and the lumen, the snare passing through the leverage member, the leverage member being configured to close the filter when the snare is retracted from outside the body before the filter is drawn into the lumen.

7. The apparatus of claim 1, wherein the frame comprises a generally cylindrical ring defining the inlet.

8. The apparatus of claim 7, wherein the frame further comprises a generally cylindrical ring defining the outlet.

9. The apparatus of claim 8, wherein the frame further comprises a plurality of struts between the rings defining the inlet and the outlet.

10. The apparatus of claim 1, wherein the sheath has an inner diameter of 18 French or less.

11. The apparatus of claim 10, wherein the outlet of the filter has an inner diameter that is greater than the outer diameter of the sheath.

12. The apparatus of claim 10, wherein the outlet of the filter has an inner diameter that is greater than an inner diameter of the sheath.

13. The apparatus of claim 1, the filter having a compressed state and a deployed state, the apparatus further comprising a guidewire and an introducer for use in percutaneously introducing the filter and the distal end of the sheath into the aortic arch, the introducer comprising:
a tapered distal nose;
a proximal end;
a guidewire lumen configured to receive the guidewire; and
a recess between the distal nose and the proximal end, the recess being configured to receive the filter in the compressed state,
the introducer being configured for insertion within the lumen at the distal end of the sheath and to retain the filter in the compressed state within the recess and between the introducer and the sheath,
the introducer, the filter, and the distal end of the sheath being percutaneously introducible into the aortic arch by pushing the introducer over the guidewire via the sheath.

14. The apparatus of claim 13, further comprising a control wire coupled to the introducer, the control wire configured to retain the introducer in place while the sheath is retracted, such refraction of the sheath allowing the filter to expand from the compressed state to the deployed state.

15. The apparatus of claim 14, the introducer being retrievable through the outlet of the filter and the lumen of the sheath after the filter expands to the deployed state by retracting the control wire.

16. The apparatus of claim 13, wherein a portion of the sheath is pre-curved to conform to the aortic arch, wherein the introducer straightens the pre-curved portion of the sheath when inserted therein.

17. A method of filtering emboli during a percutaneous aortic valve replacement or repair procedure, the method comprising:
providing a sheath having proximal and distal ends and a lumen therebetween;
providing a filter having a compressed state and a deployed state, the filter having a frame and an emboli-filtering mesh attached to the frame, the frame having an inlet and an outlet, the inlet being configured to substantially span the aortic arch in a region between the aortic valve and the great arteries in the deployed state;
percutaneously introducing the distal end of the sheath into the aortic arch;
expanding the filter from the compressed state to the deployed state within the aortic arch; and
coupling the outlet of the filter to the distal end of the sheath within the aortic arch without leaving any gaps through which emboli could pass and without obstructing the lumen at the distal end of the sheath.

18. The method of claim 17, wherein said coupling comprises drawing the outlet of the filter into contact with the distal end of the sheath by retracting a plurality of tensioning lines coupled to the frame of the filter and passing out of the body through the lumen.

19. The method of claim 17, further comprising retracting the filter into the lumen with a snare coupled to the frame of the filter and passing out of the body through the lumen.

20. The method of claim 17, further comprising:
providing a guidewire and an introducer having a guidewire lumen configured to receive the guidewire, the introducer having a recess configured to receive the filter in the compressed state;
compressing the filter to the compressed state within the recess of the introducer;
inserting the introducer into the lumen at the distal end of the sheath and retaining the filter in the compressed state within the recess and between the introducer and the sheath; and
percutaneously introducing the introducer, the filter, and the sheath into the aortic arch by pushing the introducer over the guidewire via the sheath.

21. The method of claim 20, wherein said expanding comprises retaining the introducer in place while retracting the sheath so as to expose the filter and allow the filter to expand from the compressed state to the deployed state.

22. The method of claim 21, further comprising retrieving the introducer through the outlet of the filter and the lumen of the sheath after the filter expands to the deployed state.
